(19) United States
(12) Patent Application Publication
(10) Pub. No.: US 2016/0228177 A1
(43) Pub. Date: Aug. 11, 2016

(54) A SYSTEM FOR PERIODONTAL TREATMENT

(71) Applicant: SYNERON MEDICAL LTD., Yiqbean Illit (IL)

(72) Inventors: Shimon ECKHOUSE, Haifa (IL); Tuvia Dror KUTSCHER, Shoham (IL)

(73) Assignee: SYNERON MEDICAL LTD., Yoqnearn Illit (IL)

(21) Appl. No.: 14/907,684

(22) PCT Filed: Aug. 18, 2014
(86) PCT No.: PCT/IL2014/000040

§ 371 Date: Jan. 26, 2016

Related U.S. Application Data
(60) Provisional application No. 61/875,113, filed on Sep. 9, 2013.

(51) Int. Cl. A61B 18/14; A61C 5/14; A61C 19/06; A61B 18/12

(52) U.S. Cl. CPC A61B 18/14; A61B 18/1206; A61C 5/14; A61C 19/06; A61B 2018/00321

(57) ABSTRACT

An oral treatment device for treating gingival hyperpigmentation including an applicator configured to accommodate at least one dental arcade and gingival tissue associated therewith, having a plurality of voltage to gingiva RF voltage-applying elements arranged in a matrix on one or more walls of the applicator and configured to apply to the gingiva ablative voltage and deepithelialize the gingival tissue. The voltage to gingiva RF voltage-applying elements are also configured to apply to the gingiva tissue, deep to the epithelium layer, sublative RF voltage configured to stimulate gingiva blood circulation and retard and sometimes even stop progression of gum recession.

### TECHNOLOGY FIELD

The method and applicator generally relate to oral treatment procedures and in particular to cosmetic gum hyperpigmentation and cosmetic gum recession treatment procedures.

### BACKGROUND

Oral cavity melanin hyperpigmentation is a relatively common phenomenon that may involve any portion of the oral cavity. Gingival hyperpigmentation (GHP) commonly referred to as black or dark gums, involves the gingival tissue and results from melanin granules produced by melanoblasts and deposited mainly in the basal layer and suprabasal layer of the gingival epithelium.

GHP is well documented in the literature and is considered to be multifactorial. Whether physiological or pathological GHP can be caused by a variety of local and or systemic factors. In some populations GHP is considered a genetic trait and is also known as physiological gingival pigmentation.

GHP is observed in the gingiva closest to the teeth, often as dark (brown to black) patches. Hence, the pigmented appearance of GHP may, in some people, be a source of aesthetic self-consciousness and in some cases even embarrassment. GHP is especially noted in people with a high smile line and in some instances may prevent such people from smiling.

Depigmentation is commonly treated by deepithelization or abrasion of heavily pigmented gingival areas. Scalpel surgery, gingivectomy with free gingival autografting, electrosurgery, cryosurgery, chemical agents such as 90% phenol and 95% alcohol, abrasion with diamond bur, Nd:YAG laser, semiconductor diode laser, and CO2 laser have been employed for removal of melanin hyper pigmentation (Treatment of gingival hyperpigmentation with rotary abrasive, scalpel, and laser techniques: A case series. M. Bhanu Murthy et al., J Indian Soc Periodontol. 2012 October-December; 16(4): 614-619).

The procedures remove the outer layer of the gingival epithelium with a layer of the underlying connective tissue. The new epithelium that forms is devoid of melanin. Immediate results may be observed as early as the day of the procedure itself.

The article Gingival Pigmentation Reduction: A Novel Therapeutic Modality (H. V. Mahesh et al, J Cutan Aesthet Surg. 2012 April-June; 5(2): 137-140) describes the use of radiofrequency (RF) as a clinically valuable, safe and effective method to reduce pigmentation of gingiva, disclosing that electrically generated thermal energy influenced the molecular disintegration of melanin cells that are present on the basal and supra basal cell layer of operated gingival sites. And the latent heat of radiosurgery had some influence on retarding the development and migration of melanocytes, which makes use of radiofrequency more efficient in surgical depigmentation procedure compared to conventional procedures.

Gingival (gum) recession is becoming a prominent condition in oral health. Several causes of gum recession have been identified among which are abnormal tooth position, hereditary traits resulting in thin, fragile or insufficient gingival tissue, aggressive or excessive tooth brushing, trauma to the gingival tissue and periodontal disease.

Treatment of gingival recession is primarily surgical creating a gingival flap or employing tissue grafts. The key factor in gingival recession is prevention and/or delay or arrest of gum recession already in progress. Among others, gum massage and brushing stimulates gingival tissue and increase blood flow to the tissue reducing inflammation and contributing to tissue health.

Fractional resurfacing or rejuvenation treatment is a skin ablative technology described in U.S. Pat. Nos. 6,148,232, No. 8,216,216, No. 8,357,150, and No. 8,496,654 assigned to the same assignee.

There are mainly two types of devices used to ablate the skin: laser based devices and RF based devices. Both types of these devices ablate a pattern of extremely small diameter shallow holes or zones. The holes are microscopically small treatment zones surrounded by untreated skin areas. The treatment results in a very rapid healing or recovery and skin resurfacing of the treated zone. In the healing process of the treated zones, a layer of new skin appears, restoring a fresh, youthful complexion.

The pattern of small holes is typically produced by an X-Y scanning laser beam or by application of RF energy or voltage. The laser is focused on the skin and usually operates in pulse mode ablating micron size holes in the skin.

RF based fractional skin treatment produces a scanning pattern of micron size holes in the skin a similar to laser. Typically, the voltage is delivered to the skin by an applicator equipped by a tip having a plurality of voltage to skin applying/delivering elements or contact elements arranged in a matrix or in an array. The voltage to skin applying elements are placed in contact with the segment of the skin to be treated and driven by a source of suitable power and frequency RF voltage. Application of a high voltage RF pulse to the electrodes ablates the skin under the respective electrode forming a small wound.

### SUMMARY

The instant patent application discloses a fractional deepithelization technology for cosmetic gingiva hyperpigmentation removal and gum recession.

There is therefore provided a gingiva cosmetic treatment applicator shaped as a well or channel configured to be snapped-on a dental arcade. The well or channel are formed by a front wall, rear wall and bottom. The front wall of the channel includes an inner surface (a surface in contact with the gingiva) including miniature (microscopic) discrete voltage to gingiva RF voltage application elements arranged along a strip located on one or more of the walls in a one dimensional or two-dimensional array and configured to deliver to gingival epithelium RF voltage sufficient to ablate the epithelium and bring about a removal of gingival epithelium (i.e., deepithelize).

In another example of the gingiva cosmetic treatment applicator the back wall of the gingiva cosmetic treatment applicator could be replaced with resilient retainers configured to hold the applicator on the dental arcade.

In yet another example, the retainers consist of a soft and pliable cushion positioned on top a resilient leg and configured to conform to the morphology of the gingival tissue accommodated in applicator without undue pressure on the gingiva increasing the comfort of the user.

In still another example, miniature discrete voltage to gingiva RF delivering elements could be disposed on a detachable and disposable substrate including RF supply conductors.

In another example, a gingiva cosmetic treatment applicator can include portable unit incorporated inside a comfortable to use handle having one or more activating switches as well as one or more visual indicators. The portable unit could include a power source, an RF voltage generator, a computer and a liquid or gel reservoir. The applicator 500 also includes a stem one end of which attached to the handle and the opposite end attached to a carrier including miniature discrete voltage to gingiva RF delivering elements.

In yet another example, an applicator could be a self-adhering disposable patch connected via a harness to a portable unit and including miniature discrete voltage to gingiva RF voltage application elements arranged along a strip and configured to deliver to gingival epithelium RF voltage sufficient to ablate the epithelium and bring about a gingiva deepithelization effect Gingiva treatment.

In still another example, the inner surface of the applicator could be coated with a biocompatible adhesive so that to be easily temporarily adhered to the surface of any gingival tissue, for example using a finger and be removed once treatment is completed.

In another example, the applicator could conform to the surface morphology of any desired gingival surface and be shaped and sized so that to be easily adhered to segments of gingiva to be treated such as, for example, sized to cover only pigmented patches on the gingiva, which an operator may desire to remove.

In yet another example, the apparatus is configured to generate a linear sweeping heating and/or ablating front that advances similar to an ocean wave termed “a sweeping linear wave-front effect” allowing temporal differentiation of epithelium ablation of consecutive gingival tissue segments, allowing one segment to cool before applying ablative RF voltage to a consecutive segment, reducing discomfort to the user.

In still another example, the apparatus is configured to apply a non-invasive treatment in which the miniature discrete RF voltage to gingiva application elements do not penetrate the gingiva being treated and apply non-ablative sublative RF treatment leaving the gingival epithelium intact.

### BRIEF DESCRIPTION OF THE FIGURES

FIGS. 1A is a perspective view and block view simplified illustration of an example of a gingiva cosmetic treatment applicator;

FIGS. 1B is a perspective view simplified illustration of another example of a gingiva cosmetic treatment applicator;

FIG. 2 is a perspective view and cross-section view simplified illustration of yet another example of a gingiva cosmetic treatment applicator;

FIG. 3 is a perspective view and cross-section view simplified illustration of still another example of a gingiva cosmetic treatment applicator;

FIG. 4 is a perspective view and cross-section view simplified illustration of another example of a gingiva cosmetic treatment applicator;

FIGS. 5A and 5B are perspective view simplified illustrations of two other examples gingiva cosmetic treatment applicators;

FIG. 6 is a perspective view simplified illustration of yet another example of a gingiva cosmetic treatment applicator; and

FIGS. 7A, 7B and 7C, collectively referred to as FIG. 7 are plan view and cross section view simplified illustrations of an example of use of a gingiva cosmetic treatment applicator.

### DETAILED DESCRIPTION

The term “desired depigmentation effect” as used in the present disclosure means a result of RF voltage application, which can be complete or partial removal of melanin containing epithelium.

The term “deepithelization” as used in the present disclosure means uniform removal of pigment-containing gingival epithelium.

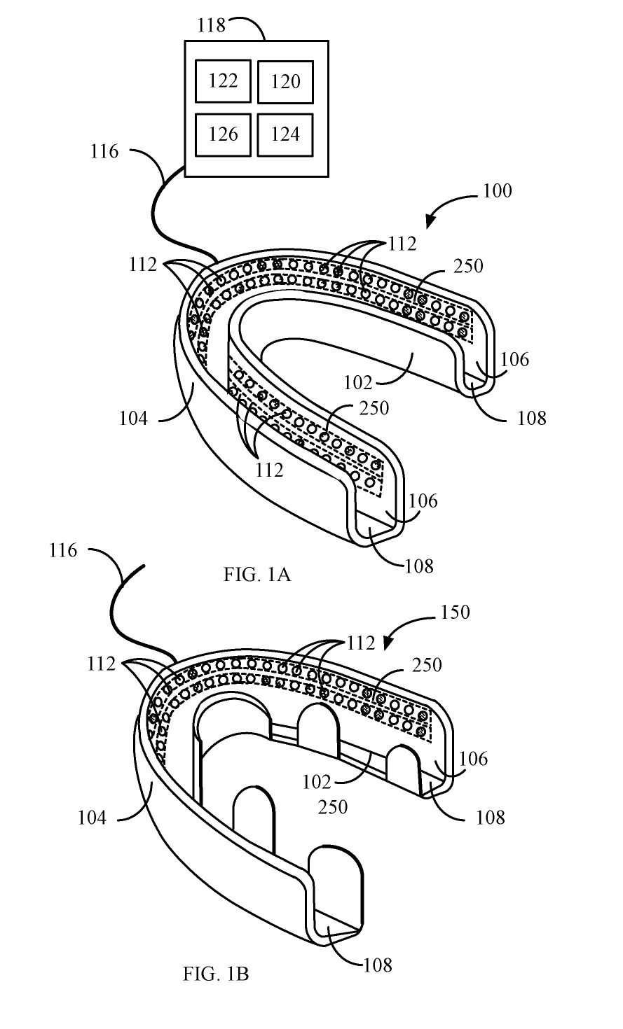

For purposes of simplification, miniature discrete voltage to gingiva RF delivering elements 112 in FIGS. 1A, 1B, 2, 3, 4, 6A, 6B and 7 are illustrated to be in two rows. However, it will be appreciated by a person skilled in the art that any number of miniature discrete voltage to gingiva RF delivering elements 112 can be arranged in any number of rows and the aforementioned figures are not intended to be limiting any way.

The present disclosure at least partially resolves the problem of gingival pigmentation by applying to discrete locations on the gingiva an RF voltage sufficient to ablate the pigment-containing epithelium layer and form a plurality of small size wounds. Healing of these wounds causes growth of a new pigmentless epithelium layer. The new epithelium layer is free of melanin and the size of pigmentation is naturally reduced or completely eliminated.

The new epithelial growth could also expand the gingival tissue and replace previously missing gingival epithelium resulting from gingiva recession.

Fractional treatment of gingiva could be as explained above performed by using a laser, however, the cost of the RF-based products is significantly lower than that of the products operating with laser radiation. Additionally, RF-based products can be made to be safer than laser-based products and to be used in a home environment by a non-professional user. Hence, RF-based gingiva (why cosmetic) treatment applicators will most probably become widely used in both clinical and home environment.

As shown in FIGS. 1A and 1B, which are perspective-view and block diagram simplified illustrations of an example of a gingiva treatment applicator, a gingiva treatment applicator 100 can, but not necessarily as will be shown below, be curved in a form of a horseshoe so that to be configured to accommodate an upper or lower human dental arcade together with at least its associated labial (front) gingival tissue. When paired, a pair of gingiva cosmetic treatment applicators 100 can be configured to accommodate both upper and lower dental arcades and their respective gingival tissue concurrently.

Gingiva cosmetic treatment applicator 100 (FIG. 1) can include a back curved wall 102 and a front curved wall 104 each having an inner surface 106, in contact with gingiva and housing RF voltage application elements configured to contact gingival tissue to be treated and being integrally attached to opposite sides of floor 108. As will be described below, gingiva cosmetic treatment applicator 100 could also include front curved wall 104 only.

Inner surfaces 106 can consist of a soft and pliable material configured to conform to the morphology of the gingival tissue accommodated in the applicator without undue pressure on the gingiva increasing the comfort of the user.

Inner surfaces 106 of one or both walls 102/104 can include miniature discrete voltage to gingiva RF voltage application elements 112 arranged along a strip 250 and configured to deliver to gingival epithelium RF voltage sufficient to ablate the epithelium and bring about a removal of gingival epithelium (i.e., deepithelize).

Applicator 100 of FIG. 1A, having inner surfaces 106 of both back curved wall 102 and a front curved wall 104 could be suitable for treatment or prevention of gingival tissue (gum) recession as will be explained in greater detail below though can be used for gingiva de-pigmentation procedures as well. Cosmetic treatment applicator 100 can be sufficiently deep so that when a dental arcade is accommodated in applicator 100, voltage to gingiva RF voltage application elements 112 contact only the labial (front) and/or lingual (back) gingiva.

Additionally and optionally, voltage to gingiva RF voltage application elements 112 can be located on front curved wall 104 only so that to treat, for example, GHP (dark areas of pigmentation) on the labial (front) maxillary or labial (front) mandibular gingiva, which are the only gingival aspects visible when smiling.

Gingiva cosmetic treatment applicator 100 can be connected via a harness 116 to a portable unit 118 that can include one or more of a power source 120, an RF voltage generator 122, a computer 124 and a liquid or gel reservoir 126. Power source 120 could be, for example, a standard electrical AC grid outlet or a battery configured to supply RF voltage generator 122. Liquid or gel reservoir 126 could store electrical conductive gel or liquid to improve contact between voltage to gingiva RF voltage application elements 112 and the gingival tissue when necessary (e.g., dry gums).

FIG. 1B shows another example of an applicator 150 for cosmetic treatment of gingival hyperpigmentation (GHP) cosmetic treatment. In the configuration depicted in FIG. 1B, voltage to gingiva RF voltage application elements 112 can be located on front curved wall 104 only so that back curved wall 102 could be replaced with one or more retainers 130 that could be semi-rigid or resilient and project from and at an angle normal to floor 108 opposite to inner surface 106 and apply pressure on the non-treated [e.g., lingual (back)] gingiva and retain the treated dental arcade in place.

Similar to inner surfaces 106 of FIG. 1A, retainers 130 can also consist of a soft and pliable material configured to conform to the morphology of the accommodated gingival tissue without undue pressure on the gingiva increasing the comfort of the user.

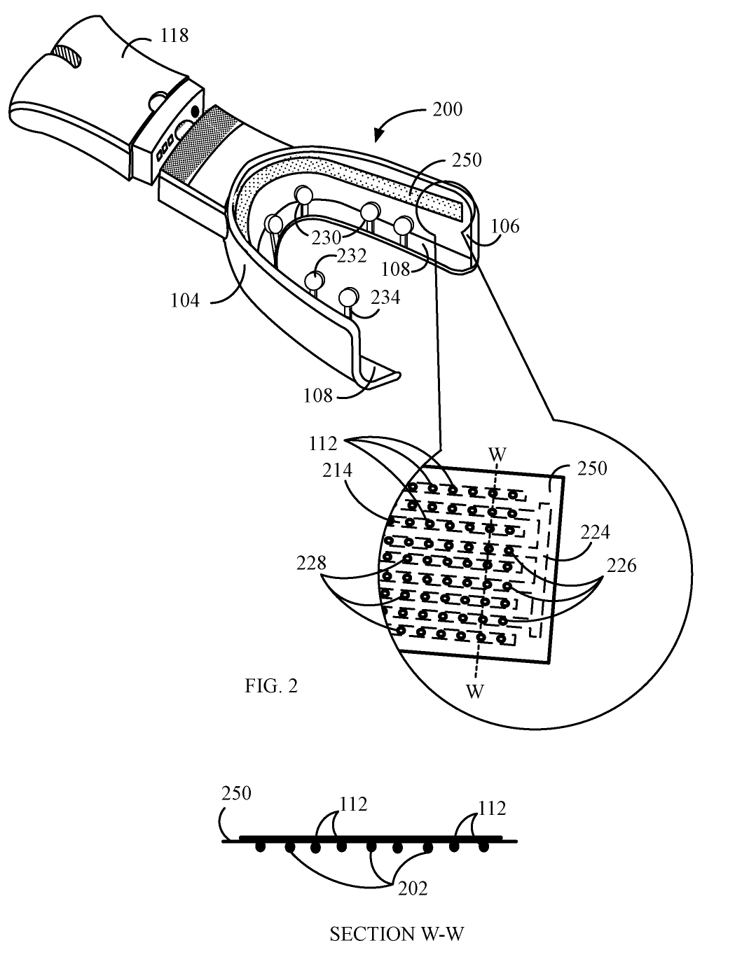

Reference is now made to FIG. 2, which is a perspective view and cross-section view simplified illustration of yet another example of a gingiva cosmetic treatment applicator. In gingiva cosmetic treatment applicator 200 back curved wall 102 could be replaced with resilient retainers 230 that project from and at an angle normal to floor 108. Resilient retainers 230 can consist of a soft and pliable cushion 232 positioned on top a resilient leg 234 and configured to conform to the morphology of the gingival tissue accommodated in applicator 200 without undue pressure on the gingiva increasing the comfort of the user. Any other type of suitable configuration of retainers 130 (FIG. 1B) and 230 can be used.

Inner surface 106 of wall 104, in contact with gingiva, can also include an array or matrix of miniature (microscopic) discrete voltage to gingiva RF voltage application elements 112. The discrete voltage to gingiva RF voltage application elements 112 could slightly protrude from inner surface 106 to facilitate a better contact with the gingiva. The discrete voltage to gingiva RF voltage application elements 112 are configured/configured to deliver to gingival epithelium RF voltage sufficient to ablate the epithelium and bring about gingiva deepithelization.

Alternatively and optionally, voltage to gingiva RF voltage application elements 112 can be configured to deliver non-ablative sublative RF voltage to gingival tissue, deep to the epithelium, so that to stimulate blood circulation in the gingival tissue and retard or sometimes stop gum recession as will be explained in greater detail below.

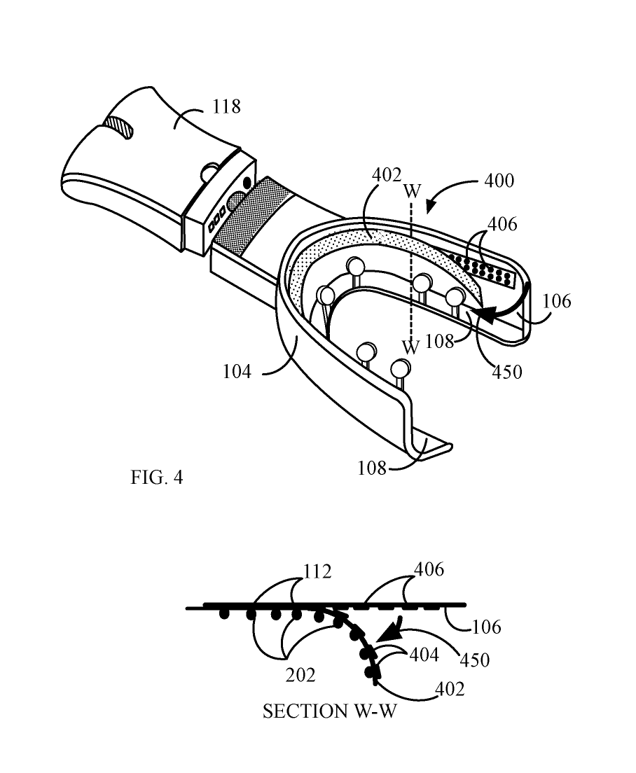

A pattern of conductors 214 and 224 shown in broken lines arranged on a strip 250 adhered to or just below inner surface 106 of wall 104 facilitate the addressing of all voltage to gingiva RF voltage application elements 112, a cluster of elements 112, or each of elements 112 individually. An arrangement of radiofrequency (RF) voltage contact points (404/406, FIG. 4) supporting connection to radio frequency voltage generator 122 (FIG. 1) can be provided by forming on or just below inner surface 106 of wall 104 contact points 406 (FIG. 4) for strips 250 communicating with radio frequency voltage generator 122 via harness 116. Voltage to gingiva RF delivering elements 112 can be arranged in a symmetrical pattern of rows and connected to contact strips 224/214 with all even rows 226 connected to one of the RF supply contact strips, for example in FIG. 2, strip 224 and all uneven rows 228 connected to another or second RF supply contact strip, for example in FIG. 2, strip 214.

Voltage to gingiva RF delivering elements 112 can be operated simultaneously, individually or in clusters. Additionally and optionally, voltage to gingiva RF delivering elements 112 can be flat or include an elevated tip, protruding from one or more inner surfaces 106. The diameter of the contact area of voltage to gingiva RF delivering elements 112 with gingival tissue can be about, but not limited to 30 to 500 micron. Such size of support voltage to gingiva RF delivering elements 112 is sufficient to apply to the gingiva RF voltage so that to bring about gingival epithelium ablation an tissue deepithelization. Other diameters of voltage to gingiva RF delivering elements 112 could be used suitable for a selected cosmetic treatment. Voltage to gingiva RF delivering elements 112 can protrude between 40 and 100 microns from inner surface 106, more commonly between 50 and 90 microns from inner surface 106 and most commonly between 60 and 80 microns from inner surface 106.

The cosmetic treatment described above can be used, for example, for cosmetic treatment of gingival hyperpigmentation (GHP). During gingival pigment removal procedure only a superficial cell layer is removed comprising part of the epidermis containing the basal layer of the epithelium and part of the epidermis. Since the damaged gingival surface is relatively small and involving the epithelium only, there may be no need in cooling.

For cosmetic treatments requiring cosmetic treatment of gingival tissue on both sides [i.e., labial gingiva (front) and lingual gingiva (back)] of the dental arcade such as, for example, gum recession cosmetic treatment, the system described in FIG. 2, employing voltage to gingiva RF voltage application elements 112 on a pattern of conductors 214 and 224 can be placed on inner surfaces 106 of both back curved wall 102 and front curved wall 104 of an applicator such as applicator 100 of FIG. 1A as will be explained in greater detail below.

Depending on the intensity of the voltage applied, electric current developed and duration of the application to gingiva the current may be enhanced and maintained for a time sufficient for gingival epithelium ablation. Typical duration of the application of RF voltage to the gingiva would be between 10 microseconds to 200 millisecond. Typical voltage values are between 10 volts and 100 volts. The treatment by itself is a non-invasive cosmetic treatment, since voltage to gingiva RF delivering elements or domes 202 do not penetrate the gingiva being treated.

As shown in FIG. 2, portable unit 118 can be directly attached to gingiva cosmetic treatment applicator 200 negating the need for harness 116 (FIG. 1). Portable unit 118 can be configured to implement a specific gingiva cosmetic treatment (e.g., gingiva deepithelization) protocol and could be replaced with another portable unit 118 when a different gingiva treatment (e.g., gingiva recession management) protocol is desired.

Alternatively and optionally, portable unit 118 can include one or more disposable components such as liquid or gel reservoir 126 or power source 120. Portable unit 118 can also serve as a handle and can be designed so that the user can close his or her lips comfortably around extension 254 connecting between portable unit 118 and applicator 200 during the treatment session.

Portable unit 118 can be used in any configuration of gingiva cosmetic treatment applicator including applicators 100, 200, 300, 400, 500, 550 and 600. Gingiva cosmetic treatment applicators 100, 200, 300, 400, 500, 550 and 600 can have adjustable settings, controlled automatically or manually or preset manually via portable unit 118 computer 124.

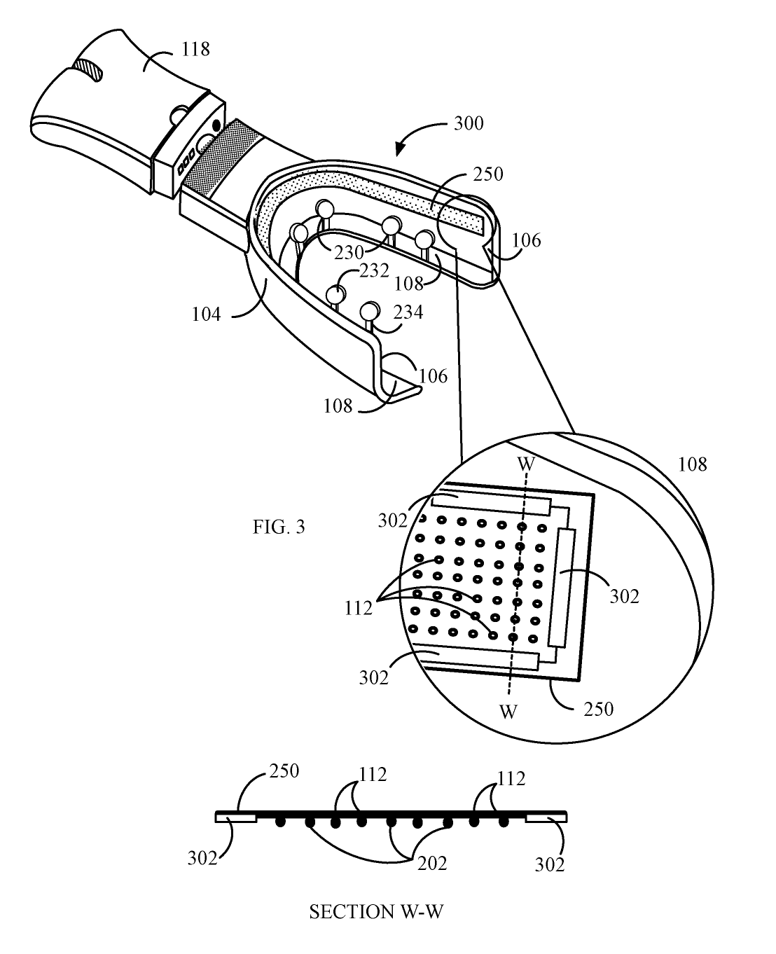

Referring now to FIG. 3, which is a perspective view and cross-section view simplified illustration of still another example of a gingiva cosmetic treatment applicator. Unlike gingiva cosmetic treatment applicator 200 which includes a symmetrical array of voltage to gingiva RF delivering elements 112 in which all elements 112 being the same, gingiva cosmetic treatment applicator 300, though also designed for a bi-polar cosmetic treatment, includes an asymmetrical distribution of voltage to gingiva RF delivering elements 112.

Gingiva cosmetic treatment applicator 300 has one or more (a cluster) large “ground” electrodes 302 located in the peripheral area of strips 250 adhered to or electrically deposited on inner surfaces 106 of walls 102/104, adjacent to or flanking voltage to gingiva RF delivering elements and connected to one or more corresponding RF supply conductors 214 or 224 (FIG. 2). All of the miniature discrete voltage to gingiva RF voltage application elements 112 slightly protruding from the substrate surface could be connected together to the other RF supply conductors 214 or 224 of strip 250.

The advantage of this solution is that the resistance variations may be more obvious since there is no competition between the electrodes located in the even and uneven contact strips, thus preventing an undesired partial imprint on the gingiva and the accompanied pain. The area of the voltage to gingiva RF voltage application elements 302 is substantially larger than the area of the terminated by dome type shapes voltage to gingiva RF voltage application elements 112.

In FIG. 4, which is a perspective view simplified illustration of still another example of a gingiva cosmetic treatment applicator, gingiva cosmetic treatment applicator 400 inner surface 106, in contact with gingiva, could include miniature discrete voltage to gingiva RF delivering elements 112 disposed on a detachable and disposable substrate 402 including RF supply conductors 214 or 224 (FIG. 2). Substrate 402 can have a first set of RF contact points 404 connected to RF supply conductors 214 or 224 (FIG. 2) via corresponding second set of RF contact points 406 on inner surface 106 of gingiva cosmetic treatment applicator 400 wall 104. Once treatment is completed, substrate 402 can be pulled or peeled away from RF contact points 406 on surface 106 as shown by arrows designated reference numeral 450 and disposed of. Surface 106 could be produced to include a small protrusion against which substrate 402 could be mounted. This would support proper positioning of contacts 404 against contacts 406.

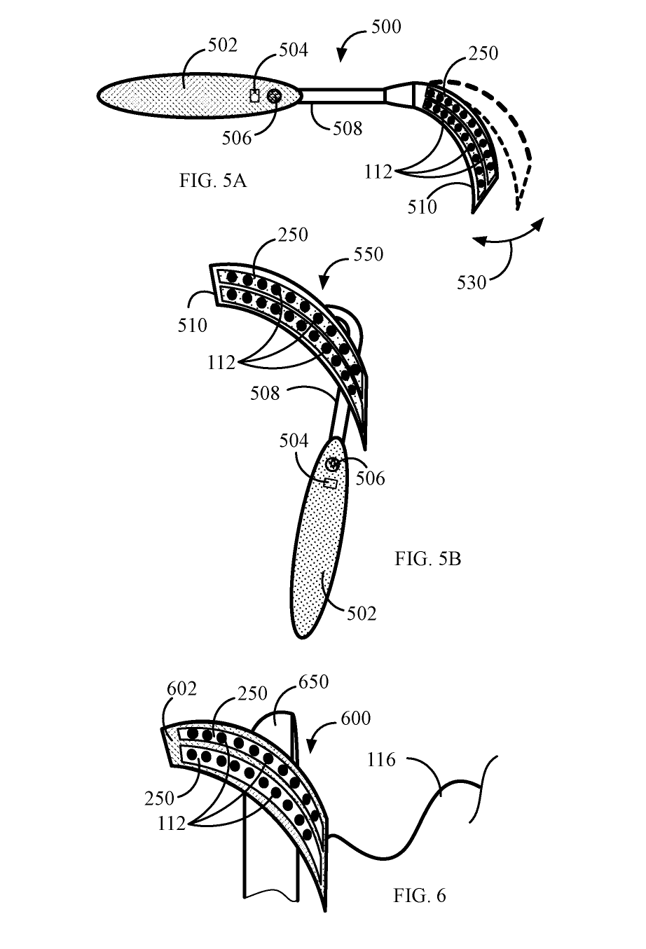

Reference is now made to FIGS. 5A and 5B, which are perspective view simplified illustrations of two examples of gingiva cosmetic treatment applicators. In the configuration of FIG. 5A, portable unit 118 is incorporated inside a comfortable to use handle 502, which also includes one or more activating switches such as, for example, on/off switch 504 as well as one or more visual indicators such as ON light 506 which illuminates when applicator 500 is activated. Applicator 500 also includes a stem 508, which could be rigid, semi-rigid or resilient for ease of application, one end of which attached to handle 502 and the opposite end attached to a carrier 510.

Carrier 510 includes strip 250 along which miniature discrete voltage to gingiva RF voltage application elements 112 are arranged and configured to deliver to gingival epithelium RF voltage sufficient to ablate the epithelium and bring about a gingiva deepithelization effect.

Miniature discrete voltage to gingiva RF voltage application elements 112 could be arranged in a configuration such as that of FIG. 2 as well as the configuration of FIG. 3 above or any other configuration suitable to bring about a gingiva deepithelization effect.

Carrier 510 could be made of a rigid material arced so that to be easily applied to the gingival surface of an upper or lower dental arcade. Alternatively and optionally, carrier 510 could be made of a semi-rigid or resilient material configured to flex in directions indicated by an arrow designated reference numeral 530 and to conform to the morphology of the gingival surface of an upper or lower dental arcade.

In another example, strip 250 of applicator 500 could be a detachable and disposable substrate similar to on a detachable and disposable substrate 402 of FIG. 4.

FIG. 5B illustrates another example of applicator 550 which is similar to applicator 500 of FIG. 5A and in which handle 502 and stem 508 attach to the center of the posterior surface of carrier 510. Applicators 500 and 550 could be suitable for cosmetic treatment of GHP in which commonly, only treatment of the labial (front) attached gingiva, which is apparent when a person smiles.

Referring now to FIG. 6, which is another example of gingiva cosmetic treatment applicator. Applicator 600 could be a self-adhering disposable patch connected via a harness 116 to a portable unit 118 as described in FIG. 1A and including miniature discrete voltage to gingiva RF voltage application elements 112 arranged along a strip 250 and configured to deliver to gingival epithelium RF voltage sufficient to ablate the epithelium and bring about a gingiva deepithelization effect Gingiva treatment.

The inner surface 602 of applicator 600, in contact with gingiva, could be coated with a biocompatible adhesive so that to be easily temporarily adhered to the surface of any gingival tissue, for example using a finger 650 and be removed once treatment is completed.

Applicator 600 could conform to the surface morphology of any desired gingival surface. Patch 602 could be also shaped and sized so that to be easily adhered to segments of gingiva to be treated such as, for example, sized to cover only pigmented patches on the gingiva, which an operator may desire to remove.

Gingiva cosmetic treatment applicator 600 could be used for cosmetic treatment of GHP as well as for cosmetic treatment of gum recession, under suitable treatment parameters, being configured to be adhered to any gingival surface including lingual gingiva, the gums on the back side of the dental arcade and be disposed of once treatment is completed.

It will be appreciated by a person skilled in the art that the gingiva (gum) treatment applicators described and illustrated above as single gingiva applicators can be fitted on either one of the upper and lower human dental arcades and gingival tissue associated therewith and can be designed to be paired so that paired gingiva treatment applicators can be fitted at the same time (concurrently) onto their respective upper and lower dental arcades thereby treating the gingiva of both dental arcades concurrently.

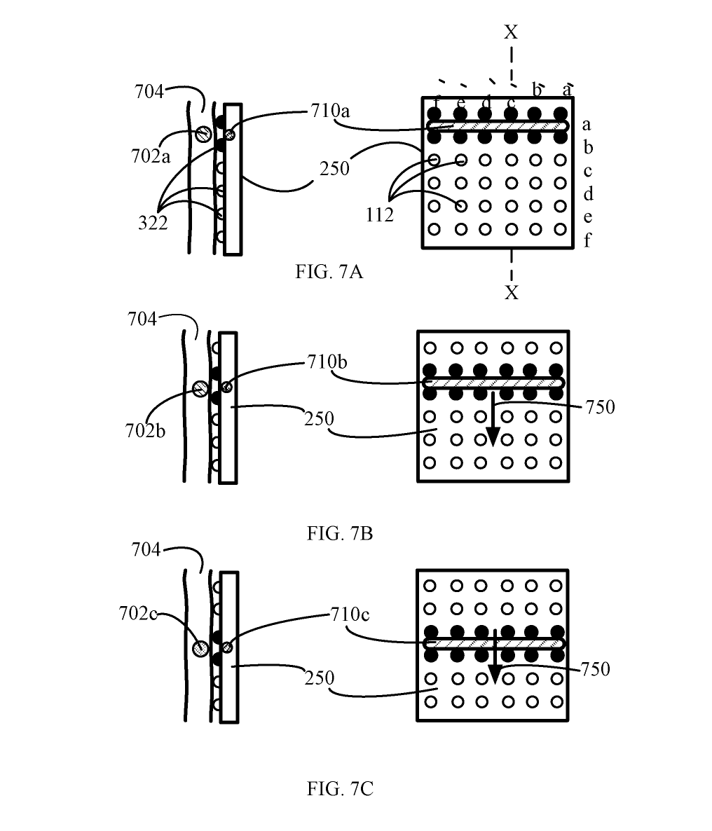

Reference is now made to FIGS. 7A, 7B and 7C collectively referred to as FIG. 7, which are plan view and cross section view simplified illustrations of an example of method of operation of a gingiva cosmetic treatment applicator such as gingiva cosmetic treatment applicators 100, 200, 300, 400, 500, 550 and 600. Each of FIGS. 7A, 7B and 7C is accompanied by a corresponding cross-sectional view taken along axis X-X (FIG. 7A).

Electrode 302, of the type shown in FIG. 2 includes a strip 250 that serves as a carrier and a plurality of miniature discrete voltage to gingiva RF delivering elements 112 in a spaced apart pattern. In FIG. 7A, elements 322 are arranged on strip 250 in an array along rows (a-f) and columns (a′-f′).

Computer 124 of portable unit 118 (FIG. 1) can be configured to activate and deactivate individual voltage to gingiva RF voltage application elements 112 arranged in FIG. 7 in an array in accordance with a predetermined treatment protocol.

As shown in FIG. 7A, only rows (a) and (b) are activated by computer 124, as indicated by the blackening of voltage to gingiva RF voltage application elements 112 of rows (a) and (b) and RF current flows from elements 112 in row (a) through gingiva tissue to be treated to elements 112 in row (b) heating and/or ablating a linear zone 702a of gingiva epithelium layer 704 therebetween and parallel thereto corresponding to an area 710a on strip 250.

As illustrated in FIG. 7B, row (a) is now inactivated and only rows (b) and (c) are activated. RF current now flows from elements 112 in row (b), through gingiva tissue to be treated, to elements 112 in row (c). Heated linear zone 702a of tissue layer 704 has now cooled down and an adjacent linear zone 702b corresponding to an area 710b on strip 250 is now heated.

In FIG. 7C, rows (a) and (b) are now inactivated and only rows (c) and (d) are activated. RF current now flows from elements 112 in row (c), through gingiva tissue to be treated, to elements 322 in row (d). Heated linear 702b zone of tissue layer 704 has now cooled down and an adjacent linear zone 702c corresponding to an area 710c on strip 250 is now heated.

This type of protocol generates a linear sweeping heating and/or ablating front that advances similar to an ocean wave, which will hereon be termed “a sweeping linear wave-front effect” allowing temporal differentiation of epithelium ablation of consecutive gingival tissue segments, allowing one segment to cool before applying ablative RF voltage to a consecutive segment, reducing discomfort to the user.

It will be appreciated by persons skilled in the art that a matrix arrangement of miniature discrete voltage to gingiva RF voltage application elements 112 enables generation of a sweeping linear wave-front effect in any desired direction, as described in U.S. Patent Application 61/307,717 to the same assignee which is hereby incorporated by reference in its entirety.

Authors of the instant patent application have found that when set at sublative cosmetic treatment parameter settings, applicators 100, 200, 300, 400, 500, 550 and 600, when activated can, under certain circumstances, apply RF voltage to gingiva RF tissue deep to the epidermis and stimulate gingiva tissue blood circulation. This contributes to the health of the gingiva tissue, can retard and sometimes even stop progression of gum recession.

Depending on the intensity of the voltage applied, electric current developed, and duration of the application to gingiva the current or arc discharge may be enhanced and maintained for a time sufficient for gingival tissue blood circulation stimulation. The parameters for cosmetic treatment of gum recession can be as follows: Typical duration of the application of RF voltage to the gingiva for cosmetic treatment of gum recession could be between 10 microseconds to 200 milliseconds. Typical voltage values for cosmetic treatment of gum recession can be between 10 volts and 1000 volts. The treatment by itself is a non-invasive treatment, since miniature discrete voltage to gingiva RF voltage application elements 112 do not penetrate the gingiva being treated. During the non-ablative sublative RF treatment procedure, the gingival epithelium is left intact.

Hence, all applications described above and the methods of their use, can be applied to gum (gingiva) recession cosmetic treatment using the above described gum recession cosmetic treatment parameters.

It will be appreciated by persons skilled in the art that the present method and system are not limited to what has been particularly shown and described hereinabove. Rather, the scope of the method and system includes both combinations and sub-combinations of various features described hereinabove as well as modifications and variations thereof which would occur to a person skilled in the art upon reading the foregoing description and which are not in the prior art.

1-41. (canceled)

42. An apparatus comprising:
at least one applicator configured to be applied to at least a portion of gingival tissue and having at least one inner surface comprising a plurality of miniature discrete voltage to gingiva RF voltage-applying elements arranged in a matrix and configured to apply to the gingival tissue ablative RF voltage and deepithelialize the gingival tissue.

43. The apparatus according to claim 42, wherein the inner surface consists of a soft and pliable material configured to conform to the morphology of the labial and/or lingual gingival tissue.

44. The apparatus according to claim 42, wherein the applicator is connected to a portable unit that includes at least one of a power source, an RF voltage generator, a computer and a liquid or gel reservoir.

45. The apparatus according to claim 42, wherein also including at least one retainer opposite to the surface and configured to apply pressure on the non-treated and retain the treated dental arcade in place and wherein the at least one retainers is resilient and consist of a soft and pliable cushion positioned on top a resilient leg and are configured to conform to the morphology of the gingival tissue accommodated in the applicator.

46. The apparatus according to claim 42, wherein the miniature discrete voltage to gingiva RF voltage application elements are microscopic and terminated by dome type shapes.

47. The apparatus according to claim 42, wherein the voltage to gingiva RF voltage application elements are adhered to a pattern of conductors that enable the addressing of all voltage to gingiva RF voltage application elements, a cluster of elements or each of the elements individually.

48. The apparatus according to claim 42, wherein the voltage to gingiva RF delivering elements are arranged in a symmetrical pattern of rows and connected to RF supply contact strips with all even rows connected to one RF supply contact strip and all uneven rows connected to another RF supply contact strip.

49. The apparatus according to claim 42, wherein the diameter of the contact area of the voltage to gingiva RF delivering elements with gingival tissue is between 30 to 500 micron.

50. The apparatus according to claim 42, wherein the duration of the application of RF voltage to the gingiva is between 10 microseconds to 200 millisecond.

51. The apparatus according to claim 42, wherein the voltage values of the RF voltage applied to the gingiva are between 10 volts and 1000 volts.

52. The apparatus according to claim 42, wherein the voltage to gingiva RF delivering elements do not penetrate the gingiva being treated.

53. The apparatus according to claim 42, wherein the applicator has at least one large “ground” electrodes adjacent to or flanking voltage to gingiva RF delivering elements and, wherein the “ground” electrode is connected to at least one corresponding RF supply conductor and the voltage to gingiva RF voltage application elements are connected together to another RF supply conductor.

54. The apparatus according to claim 42, wherein the voltage to gingiva RF delivering elements are disposed on a detachable and disposable substrate including RF supply conductors and, wherein the substrate includes a first set of RF contact points connected to RF supply conductors via a corresponding second set of RF contact points.

55. The apparatus according to claim 42, wherein the applicator comprises a handle and a stem one end of which is attached to the handle and the opposite end attached to a carrier, and wherein the carrier comprises a strip along which the miniature discrete voltage to gingiva RF voltage application elements are arranged.

56. The apparatus according to claim 1, wherein the applicator is a self-adhering disposable patch configured to be shaped and sized so that to be easily adhered to segments of gingiva to be treated.

57. The apparatus according to claim 1, wherein the applicator is configured to generate a sweeping linear wave-front effect.

58. The apparatus according to claim 1, wherein the diameter of the contact area of the voltage to gingiva RF delivering elements with gingival tissue is 30 to 500 micron and wherein the voltage to gingiva RF delivering elements protrude 40 and 100 microns from the inner surface.

59. Apparatus comprising:
at least one applicator configured to be applied to at least a portion of gingival tissue and having at least one inner surface comprising a plurality of miniature discrete voltage to gingiva RF voltage-applying elements arranged in a matrix and configured to apply to the gingival tissue non-ablative sublative RF voltage to stimulate blood circulation in the gingival tissue and retard progression of gum recession.
